(19) United States
(12) Patent Application Publication
(10) Pub. No.: US 2024/0285180 A1
(43) Pub. Date: Aug. 29, 2024

(54) FLEXIBLE PIEZOELECTRIC ARRAY FOR WEARABLE BLOOD PRESSURE SENSING

(71) Applicant: THE TRUSTEES OF DARTMOUTH COLLEGE, Hanover, NH (US)

(72) Inventors: Andrew CLOSSON, West Fairlee, VT (US); John X.J. ZHANG, Hanover, NH (US); Congran JIN, White River Junction, VT (US)

(21) Appl. No.: 18/578,900

(22) PCT Filed: Jul. 29, 2022
(86) PCT No.: PCT/US22/38897

§ 371 Date: Jan. 12, 2024

Related U.S. Application Data
(60) Provisional application No. 63/227,307, filed on Jul. 29, 2021.

(51) Int. Cl. A61B 5/021; A61B 5/00; A61B 5/024

(52) U.S. Cl. CPC A61B 5/02125; A61B 5/024; A61B 5/6807; A61B 5/6822; A61B 5/6824; A61B 5/743; A61B 2560/045; A61B 2560/0468; A61B 2562/0247; A61B 2562/028; A61B 2562/046; A61B 2562/125; A61B 2562/164; A61B 2562/166

(57) ABSTRACT

A system includes piezoelectric nanofibers, an encapsulation polymer, and patterned electrodes in an array. The array structure of the electrodes can create separate piezoelectric sensor elements, which can detect pressure from pressure such as arterial pulse waves. The sensors arrays can be used to detect the pulse wave velocity of the arterial pulse while it is superficial to near-surface arteries. This can enable detection of blood pressure in a wireless manner.

### CROSS-REFERENCE TO RELATED APPLICATIONS

This application claims priority to the provisional patent application filed Jul. 29, 2021 and assigned U.S. App. No. 63/227,307, the disclosure of which is hereby incorporated by reference.

### STATEMENT REGARDING FEDERALLY-SPONSORED RESEARCH OR DEVELOPMENT

This invention was made with government support under contract R01HL137157 awarded by the National Institute of Health. The government has certain rights in the invention.

### FIELD OF THE DISCLOSURE

This disclosure relates to flexible sensors.

### BACKGROUND OF THE DISCLOSURE

As wearable and implantable technologies for health monitoring have become more popular, functional materials such as piezoelectric and conductive polymers have been developed as building blocks for more flexible and conforming devices. Existing technology for the wearable detection of blood pressure uses LEDs and/or photodiodes to detect arterial bed volume changes.

For example, in situations of out-of-hospital cardiac arrest and its immediate care by laypersons, the ability to quickly detect the performance of adequate cardiopulmonary resuscitation (CPR) through clinically acceptable pulse rate and blood pressure (BP) is critical. However, the detection of adequate CPR can be difficult to detect for someone not trained in first aid. The gold standard for continuous BP monitoring is through insertion of an intravascular pressure sensor, but it is only available in clinical settings. Currently the standard for measuring BP noninvasively is using cuff-based oscillometric approaches that are standard at all healthcare facilities. The key issue with these cuff-based sensors is that they are inherently non-continuous, short-term measurements, and require training to use. Attempts at developing these into wearable devices for continuous measurements have proven to be difficult. The oscillometric nature of the devices causes discomfort in patients and many clinicians are worried that continuously squeezing the arteries over a long period of time will lead to damaging effects and lower accuracy measurements.

Other transduction mechanisms have been investigated for devices to detect BP in a wearable fashion including, single-lead electrocardiogram (ECG), photoplethysmography (PPG), piezoresistive, and piezoelectric systems. The most common approaches outside of oscillometeric approaches have been coupling ECG and PPG, which have non-conformable and high-power consumption (mW) mechanisms that limit their use in any sort of remote, long-term, or emergency medicine situations. In many of these approaches to cuffless BP sensing, the arterial pulse transit time (PTT) or pulse wave velocity (PWV) is detected first followed by calibration models to translate PTT/PWV to BP.

While these methods for cuffless blood pressure sensing have been investigated, two challenges remain and need to be overcome to allow for the development of a low-power flexible patch for cardiovascular monitoring during CPR. First, the current materials for the cardiovascular flow monitoring are not suitable for low power, skin conformable applications. Second, the methods for computing blood pressure from physiological signals like PTT/PWV have low accuracy due to the non-controllable nature of testing environment (e.g., a human's cardiovascular system).

Improved systems and techniques to measure pressure are needed.

### BRIEF SUMMARY OF THE DISCLOSURE

A system is disclosed in a first embodiment. The system includes piezoelectric nanofibers, an encapsulation polymer, and patterned electrodes in an array. The encapsulation polymer is configured to fill voids between the piezoelectric nanofibers. The system has a thickness from 5 μm to 1 mm. The system can further include a processor in electronic communication with the patterned electrodes and/or a wireless data transmission system in electronic communication with the patterned electrodes.

In an instance, the piezoelectric nanofibers are poly(vinylidene fluoride-cotrifluoroethylene), the patterned electrodes are poly(3,4-ethylenedioxythiophene) polystyrene sulfonate, and the encapsulation polymer is polydimethylsiloxane or parylene.

The piezoelectric nanofibers and/or the encapsulation polymer can include a dopant. The dopant can be carbon nanotubes, lead zirconate titanate (PZT), barium titanate, and/or zinc oxide.

From 90% to 100% of the voids can be filled with the encapsulation polymer.

The system can have a Young's modulus from 360-870 kPa.

The piezoelectric nanofibers, the encapsulation polymer, and the patterned electrode can be disposed on a substrate. The piezoelectric nanofibers, the encapsulation polymer, and the patterned electrode also can be configured in a core with a shell. In the core with shell example, the patterned electrodes are in the core and the piezoelectric nanofibers are disposed around the core in the shell.

The encapsulation polymer can have a thickness from 10-25 μm. The piezoelectric nanofibers can have a diameter from 10 nm to 10 μm.

In an instance, the piezoelectric nanofibers have a thickness of approximately 50 μm and the encapsulation polymer has a thickness on either side of the piezoelectric nanofibers of 15 μm.

An embodiment of the system can be used to measure blood pressure or pressure applied to the system. An applied pressure produces a strain in the piezoelectric nanofibers thereby producing a charge that is processed. A processor can process the measurements based on the charge.

### DESCRIPTION OF THE DRAWINGS

For a fuller understanding of the nature and objects of the disclosure, reference should be made to the following detailed description taken in conjunction with the accompanying drawings.

FIG. 1 shows the flexible all-polymer printed piezoelectric device. FIG. 1 (a) shows an SEM image of PVDF-TrFE nanofibers. FIG. 1(b) is a schematic showing the PDMS infill of the PVDF-TrFE nanofibers. FIG. 1(c) is a 3D schematic of a printed device structure with printed PEDOT:PSS electrode layers on the PVDF-TrFE/PDMS composite substrate encapsulated in PDMS.

FIG. 2 shows the fabrication steps of the all-polymer piezoelectric device. The first step is to electrospin PVDF-TrFE nanofibers, followed by spin coating of a filler layer of PDMS diluted in TBA, and finally, inkjet printing of conductive polymer, PEDOT:PSS, and electrode layers. The final device's electrode layer can be patterned into complex designs through this process.

FIG. 3 shows characterization of the inkjet printing of PEDOT:PSS electrodes on PVDF-TrFE/PDMS substrate. FIG. 3(a) shows PVDF-TrFE nanofibers with spin cast non-diluted PDMS on the top and PVDF-TrFE nanofibers with spin cast 1:1 PDMS:TBA dilution on the bottom. FIG. 3(b) shows PEDOT:PSS ink printed on to a standard PVDF-TrFE/PDMS substrate on the left and shows with oxygen plasma treatment and the formation of the desired film structure on the right.

FIG. 4(a) is a plot of resistance/cm of various printed layers of PEDOT:PSS. After 10 layers, the printed electrodes drop from over 1 k Ω/cm to 71 Ω/cm. FIG. 4(b) shows voltage output from a 1.25 N force of a 1 cm² PEDOT:PSS inkjet-printed device compared to a standard gold electrode device, with a schematic of each device shown in inset.

FIG. 5(a) shows voltage peak-peak output versus applied impact force on a 1 cm² device and FIG. 5(b) shows a 2×2 square array proof of concept device (schematic shown in inset). Voltage outputs from a light tapping on each element shows device functionality.

FIG. 6 shows an example arrangement of layers and inkjet printing.

FIG. 7 shows an example of an inkjet-printed PEDOT:PSS electrode.

FIG. 8 shows the flexible all-polymer printed piezoelectric device. FIG. 8(a) is an SEM image of PVDF-TrFE nanofibers. FIG. 8(b) is an SEM image of a cross-section of the PVDF-TrFE/PDMS composite where the PVDF-TrFE fibers protruding from the PDMS is evident. FIG. 8(c) is a cross-section schematic of the final printed device structure with printed PEDOT:PSS electrode layers on the PVDF-TrFE/PDMS composite substrate, encapsulated in PDMS.

FIG. 9 shows characterization of PVDF-TrFE/PDMS substrate used to allow for inkjet printing of PEDOT:PSS electrodes to form all-polymer piezoelectric sensors. FIG. 9(a) is an XRD pattern of the as-electrospun PVDF-TrFE nanofibers with a peak at 19.8° representing the piezoelectric β-phase. FIG. 9(b) is a comparison of SEM cross sectional images of PVDF-TrFE nanofibers with spin cast PDMS (top) and a 1:1 PDMS:TBA dilution (bottom). With the diluted PDMS the fiber's porous matrix is infilled without forming an additional excess PDMS layer. FIG. 9(c) is a stress-strain curve comparing the mechanical properties of the PVDF-TrFE fibers, PVDF-TrFE fibers infilled with PDMS:TBA, and pure PDMS. FIG. 9(d) is the dielectric constant of PVDF-TrFE nanofibers infilled with 4:0, 3:1, and 2:2 dilution of PDMS:TBA over a range of 20 Hz to 20 KHz. The dielectric constant increases with more dilute PDMS.

FIG. 10 shows characterization of the inkjet printing of PEDOT:PSS electrodes on PVDF-TrFE/PDMS substrate. FIG. 10(a) shows PEDOT:PSS inkjet printed on to a standard PVDF-TrFE/PDMS substrate (left) and with oxygen plasma treatment and the formation of the desired film structure (right). FIG. 10(b) shows an optical image of the lowest width PEDOT:PSS line capable with the inkjet printer/substrate on the left with the print design for testing the resolution of the printer, down to a single-drop width on the right. FIG. 10(c) plots resistance/cm of various printed layers of PEDOT:PSS. The inset image shows the printed pattern for the test electrode.

FIG. 10(d) shows testing of a 2 cm×0.25 cm PEDOT:PSS electrode resistance over 30,000 bending cycles undergoing a 1.3 cm linear deflection.

FIG. 11(a) is a voltage output from a 1.25 N force of a 1 cm² PEDOT:PSS inkjet-printed device compared to a standard gold electrode device. FIG. 11(b) shows a test to determine relationship between voltage output of the PEDOT:PSS inkjet-printed device and force. FIG. 11(c) shows testing the charge output of the device over long-term impact, which had output showing good stability. FIG. 11(d) shows testing the effect of bending on the charge output of the device from 2 mm to 12 mm of linear displacement wherein the device showed an increasing response with increasing bending length with a response of 0.687 pC/mm of linear displacement. FIG. 11(e) shows testing the charge output of the devices over long-term bending, which showed good stability but was noisier than that of the long-term impact testing.

FIG. 12(a) shows the effects of breath on the inkjet-printed device placed superficially to the carotid artery with (i) heavy breathing shows a similar waveform but higher amplitude when compared to (ii) normal breathing. In (iii) when the breath is held, the pulse waveforms become apparent and a large spike in output is seen after a gasp for breath. FIG. 12(b) shows the output from both an inhale and exhale of each breath, as well as make out the pulses from each heartbeat.

FIG. 13(a) shows a schematic showing the placements of the inkjet-printed devices for pulse detection, the left carotid artery and the right radial artery. FIG. 13(b) shows (i) a pulse signal from the carotid artery with a pulse rate of approximately 60 beats per minute, (ii) a single pulse over a 1-second time span from the dashed-box in (i) with the dicrotic notch shown in the shaded oval, (iii) pulse signal from the radial artery with an applied pressure from a wristband with a sample that is much noisier than the carotid artery signal, and (iv) a single pulse over a 1-second time span from the dashed-box in (iii). The dicrotic notch, in the shaded oval, is not as distinguishable as in in the carotid artery signal.

FIG. 14(a) shows a 2×2 square array proof of concept device (schematic shown in inset) made up of four 1 cm² square electrodes spaced 0.3 cm apart, all on the same 3 cm×5 cm PVDF-TrFE/PDMS composite substrate. Charge outputs from a light tapping on each element shows device functionality. FIG. 14(b) is charge outputs from two adjacent elements (1 and 2) as an acrylic square impacts element 2 and moves to the right until it is fully impacting element 1. Both elements show large charge output decreases while the square is directly impacting the opposite element. FIG. 14(c) shows testing shape detection of the 2×2 array by impacting elements 2, 3, and 4 with an L-shaped piece of acrylic, while element 1 remained untouched.

FIG. 15(a) is photograph of the inkjet-printed array sensor placed on a shoe sole for an athletic shoe. Each sensor element had an active area of 2 cm×5 cm. FIG. 15(b) shows testing toe-to-heel (i) and heel-to-toe (ii) movements while wearing the inkjet-printed array sensor on a shoe sole. The inset shows the labeling of the elements within the shoe sole sensor, 1-4. In (i), the time difference between the front element 4 and the back element 1 is +0.331 seconds, while in (ii) it is a −0.298 seconds, suggesting that foot strike direction can be detected. The error bars represent the standard error of the mean.

FIG. 16 shows an exemplary core-shell electrospinning model. The core-shell nanostructure shows 4.5× sensitivity to pressure when compared to standard PVDF nanofibers, which are 8.8× more sensitive than spin-cast PVDF thin films.

FIG. 17 is a schematic representation of the piezoelectric array patch placed on the brachial and carotid arteries. In FIG. 17(a), the device is made by inkjet printing conductive PEDOT electrodes on core-shell piezoelectric nanofibers. FIG. 17(b) is a TEM image of a core-shell nanofiber. FIG. 17(c) shows that arterial pulse waveforms detected by the devices can be used for measuring pulse wave velocity and blood pressure.

FIG. 18 is an exemplary wearable sensor.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

A flexible thin film that includes piezoelectric nanofibers, an encapsulation polymer, and patterned electrodes in an array is disclosed. The array is connected to a flexible printed circuit board with signal processing and wireless data transmission electronic components. The array structure of the electrodes can create separate piezoelectric sensor elements. The thin and flexible nature of the materials enables application to the skin. For example, the piezoelectric sensors can detect pressure from arterial pulse waves. The sensor arrays can be used to detect the pulse wave velocity of the arterial pulse while it is superficial to near-surface arteries. Pulse wave velocity can correlate with blood pressure and may allow detection of blood pressure in a wireless manner. In an instance, force applied at different locations along the sensor array can be detected. The embodiments disclosed herein can be used to detect heart rate when placed on the skin near the carotid and radial arteries.

Embodiments disclosed here can be sensitive to motion, which can create artifacts in the data. The nano-structured materials can be configured to increase the signal-to-noise ratio by improving the sensitivity to pressure. For a given pressure input the nano-structured material has a larger output response than that of unstructured materials, thus increasing the signal-to-noise ratio by increasing the signal.

Inkjet printing is an attractive fabrication approach in the field of flexible sensing and enables low-cost, rapid prototyping, and scalable fabrication of these devices. One of the benefits of inkjet printing is its ability to rapidly iterate through many patternable designs. Fabrication of piezoelectric devices in a patternable, array format can be used in physical biomarker sensors. Poly(vinylidene fluoride-cotrifluoroethylene) (PVDF-TrFE) can be used as a piezoelectric material. PVDF-TrFE provides good flexibility while maintaining its piezoelectric constant. Its fabrication in a nanofibrous structure provides an improved voltage output when compared to thin films.

Poly(3,4-ethylenedioxy thiophene) polystyrene sulfonate (PEDOT:PSS) is a conductive polymer that has high conductivity, ease of use, and flexibility. It has been inkjet-printed as a conductive layer, including on PVDF thin films. However, the porous nature of the PVDF-TrFE nanofiber matrix can cause leakage of the conductive layer through the fibrous matrix, leading to electrical shorting. To avoid this, the void spaces within the PVDF-TrFE nanofibers can be filled to prevent ink leakage. The technique enables the fabrication of all-polymer arrayed piezoelectric devices using inkjet printing. In an instance, thinner diameter piezoelectric nanofibers can provide improved performance. An overall thinner device also can improve how conformable the device is against, for example, human skin.

Piezoelectric and piezoresistive approaches involve inherently low power (<nW) and provide a possibility for autonomous sensing. Historically, piezoelectric ceramics such as lead zirconate titanate (PZT) have been used for pressure sensors, however, they lack the mechanical flexibility to interface with soft biological tissue. Developments in piezoelectric polymers, such as polyvinylidene fluoride (PVDF) and its copolymer PVDF-TrFE, have increased the flexibility of these devices. However, these materials tend to lack the piezoelectric response that the stiffer ceramics have, reducing the ability to detect subtle changes seen in the arteries. For this reason, improved signal outputs from piezoelectric polymer materials are needed, which can be accomplished through the use of a nanofiber coupled with inkjet printing patterning of electrodes. This can include the core-shell design or other embodiments disclosed herein.

Using nanostructured and array patterned flexible piezoelectric devices can provide ultra-sensitivity for the detection of pulsed flows, such as with continuous measurements of blood flow through arteries. In the field of flexible piezoelectric sensors, nanofiber morphologies are often used for their superior outputs. With the development of, for example, these core-shell structured materials, the outputs of these devices can increase, opening up the field to an even greater number of applications.

Cuffless BP sensing has primarily been focused on the use of PPG and ECG sensing, but piezoelectric arrays can be used for BP sensing and cardiovascular monitoring.

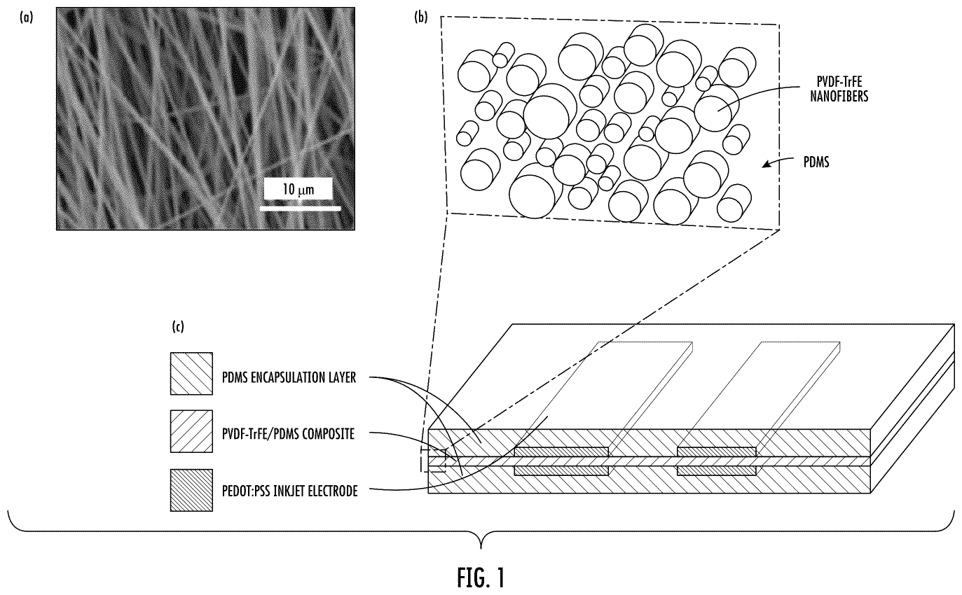

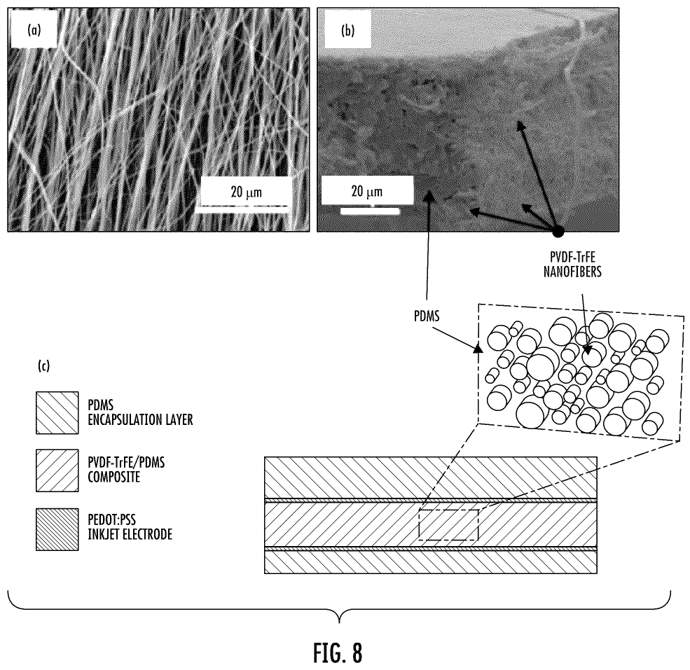

As shown in, for example, FIG. 1(c) or FIG. 8(c), the system can include piezoelectric nanofibers (e.g., PVDF-TrFE), an encapsulation polymer (e.g., polydimethylsiloxane (PDMS)) in an encapsulation layer, and patterned electrodes in an array (e.g., poly(3,4-ethylenedioxythiophene) polystyrene sulfonate (PEDOT:PSS) inkjet electrode). In an instance, the piezoelectric nanofibers can be a PVDF-TrFE/PDMS composite. The piezoelectric nanofibers can have a diameter from 10 nm to 10 μm.

Conductive material dopants can be added to the PEDOT:PSS-based fibers in a various ratios to improve the material's sensitivity. These dopants can include carbon nanotubes, metallic nanoparticles, and/or zinc oxide.

While PDMS is disclosed as the encapsulation polymer, other flexible materials that are biocompatible may be used. For example, parylene also can be used as the encapsulation polymer.

The system can have an overall thickness less than 1 mm, but larger thicknesses are possible. For example, the system can have a thickness from 5 μm to 1 mm, including all ranges and values to the 1 μm between. An overall thickness of few hundred μm is possible. This thickness can be measured from a surface that receives measurements from and/or is contact with, for example, the human body, to an opposite surface of the system.

The thickness of the piezoelectric nanofibers is at least partly determined by electrospinning time and can be from 5 μm to a few hundred μm (e.g., 5-100 μm or 5-200 μm). Thickness of the encapsulation layer is at least partly determined by the viscosity of the applied material and processing parameters. Thickness of the encapsulation layer can range from >5 μm to greater than 1 mm (e.g., 10-25 μm). The overall sensor can be configured to have a minimal thickness while remaining mechanically robust. As such thicknesses, the system can have a Young's modulus from 360-870 kPa.

In an embodiment, the piezoelectric nanofibers have a thickness of approximately 50 μm and the encapsulation polymer has a thickness on either side of the piezoelectric nanofibers of 15 μm.

The encapsulation polymer is configured to fill voids between the piezoelectric nanofibers. For example, from 90% to 100% of voids are filled with the encapsulation polymer. In an embodiment, approximately 100% of the voids are filled with the encapsulation polymer. Infilling with the encapsulation polymer allows for uniform patterning of the electrodes and can enable an all-polymer piezoelectric device.

In an embodiment, the piezoelectric nanofibers and/or the encapsulation polymer can include a dopant. The dopant can be carbon nanotubes (e.g., 0.01-3.00 weight %), lead zirconate titanate (PZT) (e.g., 1-25 weight %), barium titanate (e.g., 1-25 weight %), and/or zinc oxide (e.g., 1-10 weight %). Other metallic nanoparticles also can be added.

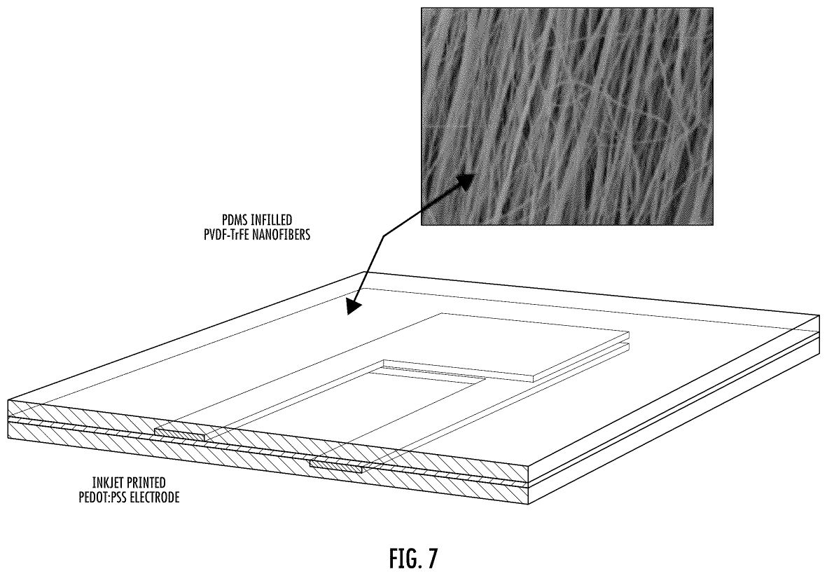

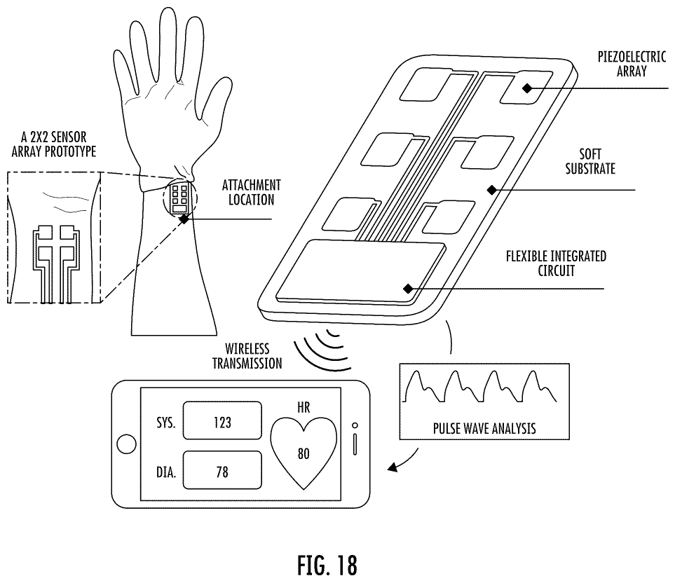

In an embodiment, the piezoelectric nanofibers, the encapsulation polymer, and the patterned electrode can be disposed on a substrate. For example, a substrate is shown in FIG. 7 or FIG. 18. PVDR-TrFE provide a flexible and conformable material. Inkjet printing provides the ability to easily pattern substrates with high throughput and scalability.

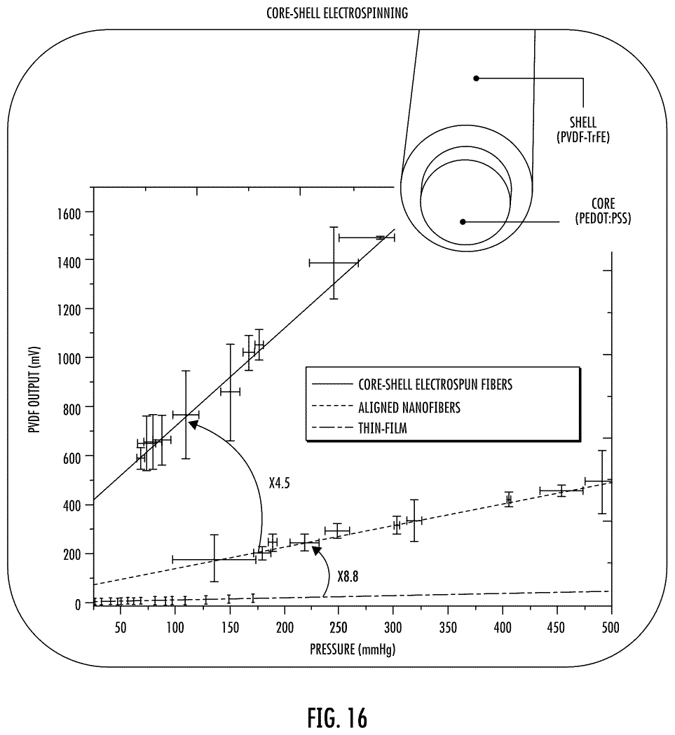

In another embodiment, the piezoelectric nanofibers, the encapsulation polymer, and the patterned electrode can be configured in a core with a shell, such as that shown in FIG. 16. For example, the patterned electrodes are in the core and the piezoelectric nanofibers are disposed around the core in the shell.

The system can include a processor in electronic communication with the patterned electrodes and a wireless data transmission system (e.g., Bluetooth) in electronic communication with the patterned electrodes. For example, an integrated circuit with a wireless data transmission system is shown in FIG. 18. A similar processor in electronic communication with the patterned electrodes and a wireless data transmission system can be used with other embodiments.

The processor typically comprises a programmable processor, which is programmed in software and/or firmware to carry out the functions that are described herein, along with suitable digital and/or analog interfaces for connection to the other elements of the system. Alternatively or additionally, the processor comprises hard-wired and/or programmable hardware logic circuits, which carry out at least some of the functions that are described herein. The processor may comprise a single functional unit or multiple, interconnected control units, with suitable interfaces for receiving and outputting the signals that are illustrated in the figures and are described in the text. Program code or instructions for the processor to implement various methods and functions disclosed herein may be stored in readable storage media, such as a memory in the processor or other memory.

Pressure applied to the system can be measured using an embodiment of the system. In an instance, blood pressure can be measured using an embodiment of the system. The applied pressure can be measured using the system and affiliated processor. An applied pressure produces a strain in the nano-structured piezoelectric material. Due to the material's inherent piezoelectric property, a charge is produced on the material's surface that is proportional to the applied pressure. The charge is converted by a charge amplifier to a voltage signal, which is then processed by a data acquisition system, such as the processor.

The following examples are provided for illustrative purposes and are not intended to be limiting.

### Example 1 (FIGS. 1-5)

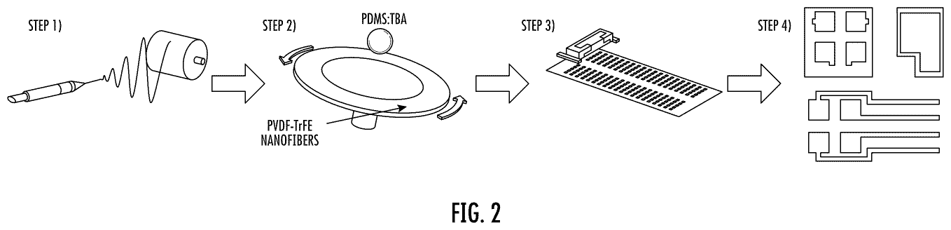

Inkjet printing on PVDF-TrFE nanofibers and the fabrication of the devices disclosed herein can be enabled by the infill of the void spaces in the PVDF-TrFE nanofibrous matrix. FIG. 1(a) shows a scanning electron microscopy (SEM) image of typical sub-micron diameter PVDF-TrFE nanofibers. The high level of porosity is evident in the image. These void spaces are infilled with a diluted PDMS solution (FIG. 1(b)), which allows for inkjet patterning of conductive polymer (PEDOT:PSS) and the formation of the basic all-polymer piezoelectric device (FIG. 1(c)). The schematic for the fabrication process of the all-polymer piezoelectric devices is demonstrated in FIG. 2, which includes step 1) electrospinning of PVDF-TrFE nanofibers, step 2) spin coating of diluted PDMS, step 3) inkjet printing of conductive PEDOT:PSS electrode layer, and step 4) PDMS encapsulation of the all polymer piezoelectric devices.

In step 1 of FIG. 2, PVDF-TrFE nanofibers were fabricated through a standard electrospinning process. A solution of 18 wt % PVDF-TrFE (70:30) was mixed into a 1:1 ratio of dimethylformamide (DMF) and methyl ethyl ketone (MEK). The solution was electrospun using a 20-gauge needle, a flow rate of 600 μl/hour, and an applied voltage of 12 kV. A rotating drum collector was used to create aligned fibers, at a distance of 9 cm from the needle, and a rotation speed of 2,000 rpm. The needle was mounted on linear motor to create a more highly uniform fiber mat. Due to the in-situ poling, no further electrical poling or thermal annealing processes were required.

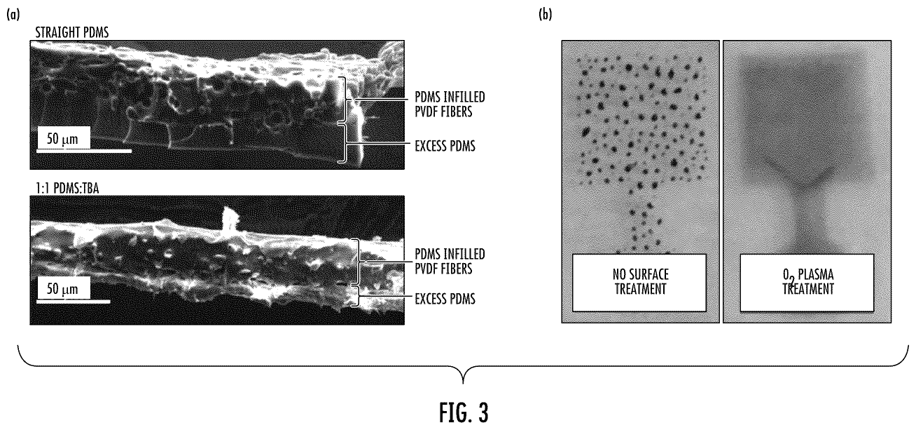

Initial attempts to print conductive layers onto the PVDF-TrFE nanofiber substrates led to electrical shorting, even in thin, single layered prints. To counteract this leakage, in step 2, a filler layer of spin-cast PDMS was used. To reduce the thickness of this filler layer and to avoid signal loss due to the increased insulating layer by increasing the resistance for charge transfer across the mat, the PDMS was diluted in tert-Butyl alcohol (TBA). 1.5 mL of a 1:1 solution of PDMS diluted in TBA was dropped onto the PVDF-TrFE nanofiber substrate and allowed to settle for 1 minute. The fibers were then spin coated for 1 minute at 5000 rpm. The PVDF-TrFE/PDMS composite was then heated in the oven for 1 hour at 60° C. to cure the PDMS. The final PVDF-TrFE/PDMS composite forms a transparent film, changing from the standard opaque white color of electrospun PVDF-TrFE nanofiber mats. Similar spin-cast PDMS layers that have been used to infill porous PVDF thin films have been shown to enhance piezoelectric output due to a higher incompressibility of the infill materials. FIG. 3(a) shows the effect that PDMS dilution in TBA has on the excess filler layer, reducing the thickness to approximately 5-6 μm. PDMS dilution reduces viscosity, which leads to the decreased thickness. For example, the thickness was approximately 5-6 μm in TBA solution compared to approximately 12 μm in straight solution.

The mechanical properties of the fiber substrate made with the diluted PDMS and the straight PDMS were compared using a tensile test (Instron). The Young's modulus of the fibers with straight PDMS was found to be 0.48 MPa, while the fibers with PDMS diluted in TBA was 0.92 MPa. The material made with diluted PDMS was more highly comprised of the stronger PVDF-TrFE nanofibers than the material made with undiluted PDMS material and, thus, had a larger Young's modulus.

The conductive polymer electrodes were inkjet-printed using a Fujifilm Dimatix DMP-2850 with 0.8 wt % PEDOT:PSS ink (Millipore-Sigma) with a drop spacing of 20 μm. The ink was first sonicated for 10 minutes at room temperature and then any aggregates were removed using a 40 μm filter. Nozzle voltage was set to 40 V and the substrate was heated to 40° C. during printing. Before printing the surface of the substrate was treated using an oxygen plasma to improve its surface properties. The effects of printing with and without oxygen plasma treatment can be seen in FIG. 3(b). Without any treatment the surface shows poor wettability. The aqueous ink forms many individual droplets and does not form the desired continuous film layer. Printing on an untreated surface can cause island formations. The O₂ plasma treatment can alter the surface energy to allow for patterning of uniform thin films.

After printing of the top electrode layer, the device was dried on a hot plate at 60° C. for 30 minutes. The device was then flipped over, and the same plasma treatment and printing steps were repeated to form the bottom electrode layer. The electrode patterning could be easily and rapidly tuned using the printer, allowing printing of any design of interest. For initial characterization, a 1 cm² square device and a small 2×2 array structure of 0.5 cm² squares were used. The entire device was finally coated in an approximately 25 μm thick layer of PDMS to serve as an encapsulation layer.

The PDMS infill procedure was developed to allow for patterning of conductive polymer electrodes by inkjet printing. The process was characterized by determining the printer's resolution, the conductivity of electrodes, and the voltage output of the devices.

The printing resolution of the electrode ink on the PVDF-TrFE/PDMS substrate was characterized using an optical microscope to measure line width in accordance with droplet width. An array of lines were printed with increasing width, starting with a width of 2 drops and increasing to 20 drops. After printing, the printed lines were imaged to show that all the printed lines remained continuous, demonstrating that printer resolution of at least a 2 drop width (approximately 200 μm) is obtained with the PEDOT:PSS ink. This gives a boundary condition to pattern development for PEDOT:PSS electrode traces of a minimum of 200 μm in thickness in one instance.

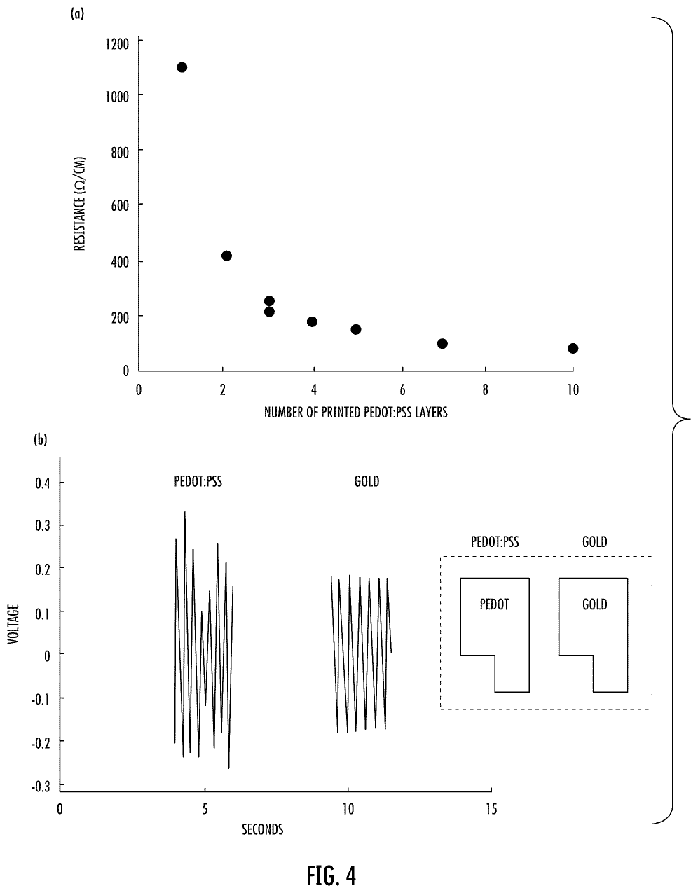

The conductivity of the printed electrodes was characterized in a per layer basis. The resistance of the 1 cm printed traces PEDOT:PSS was measured across a 1 cm gap with increasing number of printed layers (FIG. 4(a)). Each layer was allowed to dry for 5 minutes and the final printed film was dried on a hot plate at 50° C. for 30 minutes before testing using a 2-probe multimeter (Agilent). Resistance for three prints at each thickness was measured. A single layer of PEDOT:PSS had a resistance of 1,096 Ω/cm while 10 layers reached 71 Ω/cm. FIG. 4(a) shows that at 3 layers, 239 Ω/cm, the resistance no longer is decreasing as drastically with each successive printed layer, so the rest of the devices were printed using 3 layers.

To determine the viability of piezoelectric sensor devices created through this inkjet printing process, a 1 cm² square shaped electrode was printed (schematic seen in the inset of FIG. 4(b)). For the remaining tests, all device outputs were connected to a low-noise preamplifier (Stanford Research Systems) with a gain of 1 and a low-pass filter cutoff of 30 KHz. From a 1.25 N impact force at 1 Hz on the single element device reasonably large outputs of upwards of 0.5 V peak-peak were achieved (FIG. 4(b)). For comparison, a standard PVDF-TrFE nanofiber device of the same size with magnetron sputtered gold electrodes and no PDMS infill was fabricated. FIG. 4(b) shows that impact force on the inkjet-printed PEDOT:PSS device has a similar response as the standard gold electrode device, which demonstrates that the printed polymer electrodes piezoelectric devices can show performances equal to that of standard metallic electrodes piezoelectric devices.

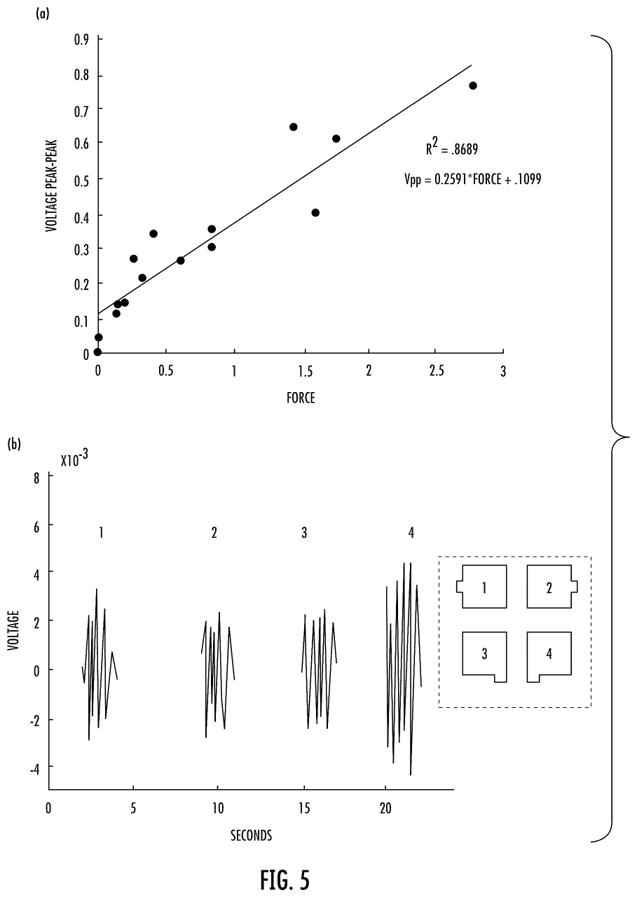

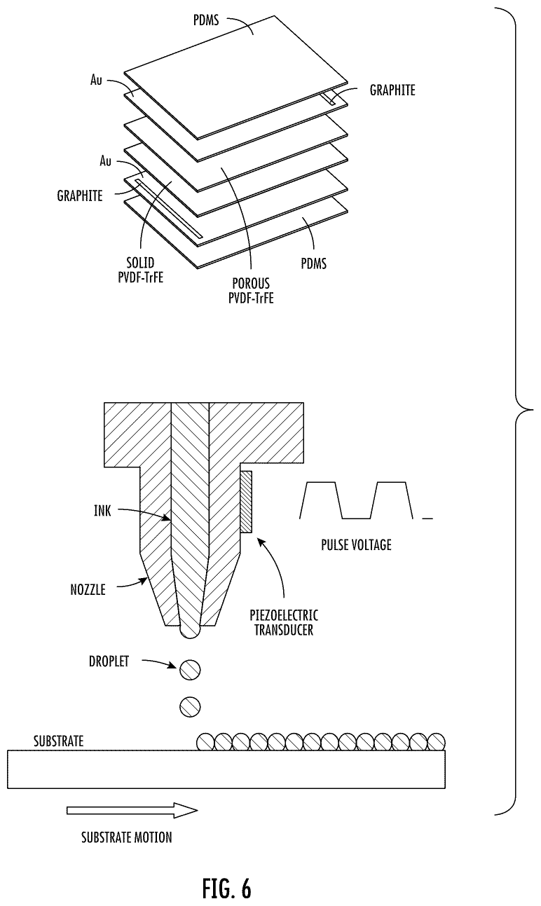

To determine the printed PEDOT:PSS single element's sensitivity to force inputs, peak-to-peak voltage outputs were measured over a variety of forces from 0-3 N from direct impacts with a 1 cm² square impact area at a frequency of 1 Hz. The testing set up included a shaker (2025E from the Modal Shop) for controlling of impact force and frequency, a force transducer (Tekscan), and a fixture frame for mounting the devices. FIG. 5(a) shows the voltage response. A linear fit gives a peak-to-peak voltage response of 259 mV/N with an R² value of 0.8689.

A fabrication method that utilizes an inkjet printer provides rapid prototyping ability and easy pattern formation. To show the application of this patterning ability, a 2×2 array was printed (schematic seen in the inset of FIG. 5(b)). The 2×2 array was made up of four 0.5 cm² squares all on the same PVDF-TrFE/PDMS composite substrate. FIG. 5(b) shows individual outputs of each element from a simple light tapping with a small insulating rod as a proof of concept, showing that each element of the device is functional. This fabrication method for inkjet printing of polymer electrodes on PVDF-TrFE nanofiber devices can provide a variety of rapid and easily fabricated patterns.

Through the addition of a physical separation layer of PDMS, inkjet patterning can be performed for PEDOT:PSS electrodes on PVDF-TrFE nanofibers to create an all-polymer piezoelectric array device. The fabrication methods were characterized, and as a proof of concept, single element and simple arrays were fabricated and characterized. The current devices showed a force sensitivity of 259 mV/N and can be used as a scalable fabrication method for sensitive and flexible piezoelectric sensors.

### Example 2 (FIGS. 7-15)

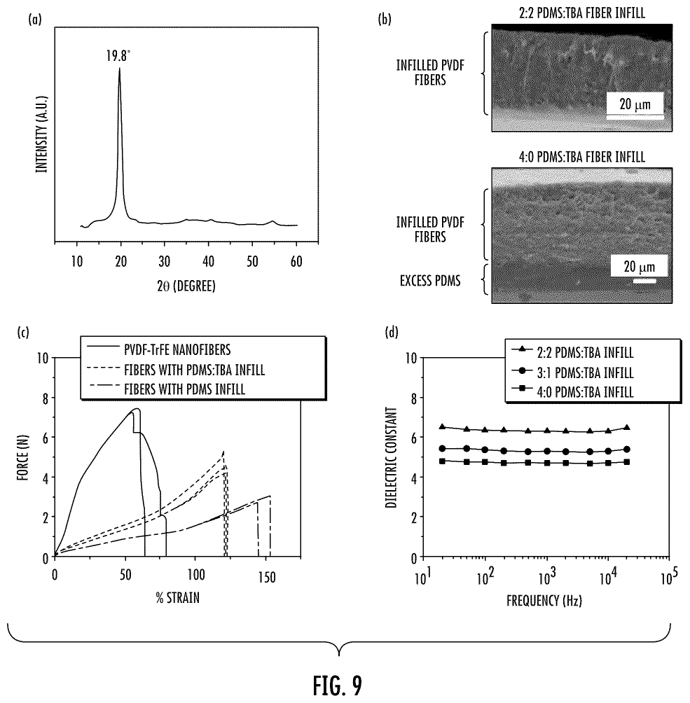

An exemplary device is shown in FIG. 7. In step 1, PVDF-TrFE nanofibers were fabricated through a standard electrospinning process. This process is similar to that illustrated in FIG. 2 or FIG. 8. A solution of 18 wt % 70:30 PVDF-TrFE (Piezotech) was mixed into a 1:1 ratio of dimethylformamide (DMF) and methyl ethyl ketone (MEK). The solution was electrospun using a 20-gauge needle, a flow rate of 600 μl/hr, and an applied voltage of 12 kV. A rotating drum collector was used to create aligned fibers at a distance of 9 cm from the needle, and a rotation speed of 3,000 rpm. The needle was mounted on a linear motor to create a more highly uniform fiber mat. Due to the in-situ poling, no further electrical poling or thermal annealing processes were performed. The electrospinning process formed fibers from 500 nm to 1 μm in diameter, with large bulk alignment. The as-electrospun nanofibers were characterized using X-ray diffraction (Rigaku) in FIG. 9. The key peak at 19.8° represents the piezoelectric β-phase of the semi-crystalline polymer, PVDF-TrFE.

Initial attempts to print conductive layers onto the PVDF-TrFE nanofiber substrates to form electrode-piezoelectric-electrode sandwich structured devices led to electrical shorting across the parallel electrodes. This shorting occurred even in thin, single layered prints. The electrical shorting was due to the leakage of the ink through the porous structure of the PVDF-TrFE fiber matrix. To counteract this leakage, in step 2, a filler layer of spin-cast PDMS was used.

To reduce the thickness of this filler layer and to avoid signal loss due to the increased insulating layer by increasing the resistance for charge transfer across the mat, the PDMS was diluted in tert-Butyl alcohol (TBA). 1.5 mL of a 4:0, 3:1, and a 1:1 solution of PDMS diluted in TBA was dropped onto the PVDF-TrFE nanofiber substrate and allowed to settle for 1 minute. The fibers were then spin coated for 1 minute at 5000 rpm. The PVDF-TrFE/PDMS composite was then heated in the oven for 3 hours at 60° C. to cure the PDMS. The final PVDF-TrFE/PDMS composite forms a transparent film, changing from the standard opaque white color of electrospun PVDF-TrFE nanofiber mats. Similar spin-cast PDMS layers that have been used to infill porous PVDF thin films have been shown to enhance piezoelectric output due to a higher incompressibility of the infill materials. FIG. 9(b) shows the effect that PDMS dilution in TBA has on the excess filler layer by examining SEM cross section images of fibers infilled with non-diluted (4:0) and diluted (2:2) PDMS:TBA dilution. The SEM cross sectional image of the diluted PDMS:TBA infill shows a fiber matrix that has been filled in to form a solid film with embedded fibers. In the SEM cross sectional image of the non-diluted PDMS, an additional PDMS film layer of approximately 20 μm forms. This is due to the increased viscosity of the non-diluted PDMS. In the 2:2 PDMS:TBA dilution the decrease in viscosity leads to thinner films of PDMS during spin coating. The measured thickness of the fibers infilled with 4:0, 3:1, and 1:1 PDMS:TBA solutions were 75, 60, 45 μm, respectively.

The mechanical properties of the fiber substrate made with the diluted PDMS were investigated using a tensile test (Instron), the results are shown in FIG. 9(c). The tensile extension curves are also compared with that of the PVDF-TrFE fiber matrix and a pure PDMS thin film of similar thickness. The material made with diluted PDMS was more highly comprised of the stronger PVDF-TrFE nanofibers than the pure PDMS material and thus had a larger Young's modulus. Due to the addition of the high-strain capable PDMS infill, the composites were able to undergo much larger strains than that of the regular PVDF-TrFE fiber matrix. Using the lower viscosity PDMS dilution as an infill also affects the electrical properties of the infilled fibers. The dielectric constant of the fibers infilled with 4:0, 3:1, and 2:2 PDMS:TBA dilution were measured over a frequency range of 20 Hz-20 KHz (IET Labs). The 2:2 dilution had a dielectric constant of 6.32, decreasing to 5.34 and 4.72 for the 3:1 and 4:0 dilutions, respectively. PVDF-TrFE typically has a higher dielectric constant of 13, and the additional thicker layers of PDMS decreases the composites to approach that of pure PDMS of 2.4.

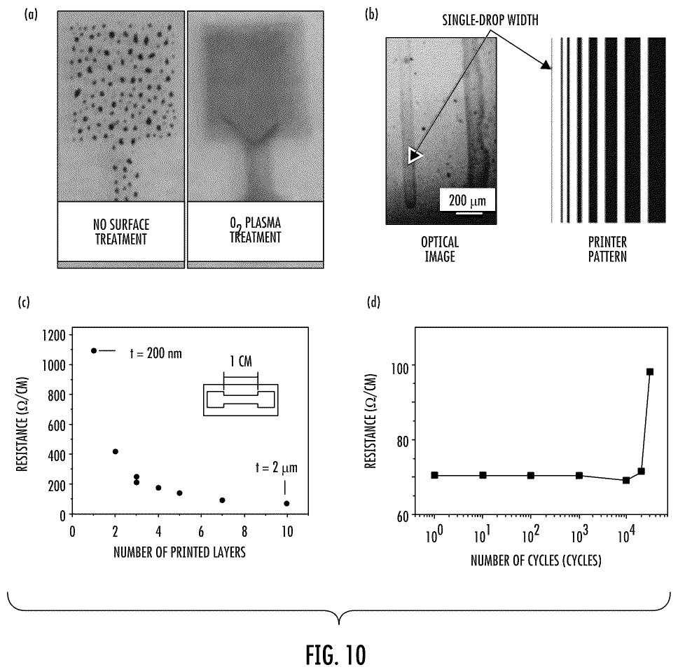

The conductive polymer electrodes were inkjet printed using a Fujifilm Dimatix DMP-2850 and a 10 pL nozzle head with 0.8 wt % PEDOT:PSS ink (Millipore-Sigma) with a drop spacing of 20 μm. The ink was first sonicated for 10 minutes at room temperature and then any aggregates were removed using a 40 μm filter. Nozzle voltage was set to 40 V and the substrate was heated to 50° C. during printing. Before printing, the surface of the substrate was treated using an oxygen plasma (Harrick Plasma) to improve its surface properties. A handheld laboratory corona discharge treater also was successful (Electro-Technic Products). The effects of printing with and without oxygen plasma treatment can be seen in FIG. 10(a). Without any treatment the surface shows poor wettability and the aqueous ink forms many individual droplets and does not form the desired continuous film layer. After printing of the top electrode layer, the device was dried on a hot plate at 60° C. for 30 minutes. The device was then flipped over, and the same plasma treatment and printing steps were repeated to form the bottom electrode layer. The electrode patterning could be easily and rapidly tuned using the printer, allowing printing of any design of interest. For initial characterization, a 1 cm² square device and a 2×2 array structure of 1 cm² square elements were used. The entire device was finally coated in a layer of PDMS to serve as an encapsulation layer.

The PDMS infill procedure was developed to allow for patterning of conductive polymer electrodes by inkjet printing on piezoelectric polymer nanofibers. The process was characterized by determining the printer's resolution, and the conductivity and robustness of printed electrodes.

The printing resolution of the electrode ink on the PVDF-TrFE/PDMS substrate was characterized using an optical microscope to measure line width in accordance with droplet width. An array of lines was printed with increasing width, starting with a width of 1 drop and increasing to 20 drops. After printing the printed lines were imaged to show that all the printed lines remained continuous, demonstrating that with the PEDOT:PSS ink a printer resolution of at least a 1 drop width (approximately 100 μm) can be obtained, as seen in FIG. 10(b). This gives a boundary condition to pattern development, with PEDOT:PSS electrode traces to be a minimum of 100 μm in width.

The conductivity of the printed electrodes was characterized in a per layer basis. The resistance of the printed PEDOT:PSS was measured across a 1 cm gap with increasing number of printed layers in three separate printed electrodes (FIG. 10(c)). Each layer was allowed to dry for 5 minutes and the final printed film was dried on a hot plate at 60° C. for 30 minutes before testing using a 2-probe multimeter (Agilent). The thickness of each additional printed layer was found to be 200 nm using interference microscopy (ADE Phase Shift MicroXAM-100 Interferometric Surface Profiler), for a total thickness of 2 μm in a ten-layer electrode. Each layer adds 200 nm to the electrode structure. After ten layers, the printed electrodes drop from over 1 kΩ/cm to 71 Ω/cm. The single layer of PEDOT:PSS had a resistance of 1,096 Ω/cm while ten layers reached 71 Ω/cm. FIG. 10(c) shows that at five layers, 146.2±1.3 Ω/cm, the resistance no longer is decreasing as drastically with each successive printed layer, so the rest of the devices that were printed for testing used five-layer electrodes.

The robustness of the printed electrodes was tested under long-term cyclic bending in FIG. 10(d). A 2 cm×0.25 cm electrode was mounted to a stepper motor and underwent a 1.3 cm linear deflection at a rate of 0.5 Hz. The resistance was measured periodically and the average percentage change in resistance across the 2 cm electrode was plotted against the number of bending cycles. In the first 10,000 cycles there is little change in resistance. The resistance began to increase after 30,000 cycles after which it was considered that the device had failed, and measurements stopped. The cyclic testing shows as a proof-of-concept that the printed polymer electrodes can undergo many deformations without severe degradation in performance.

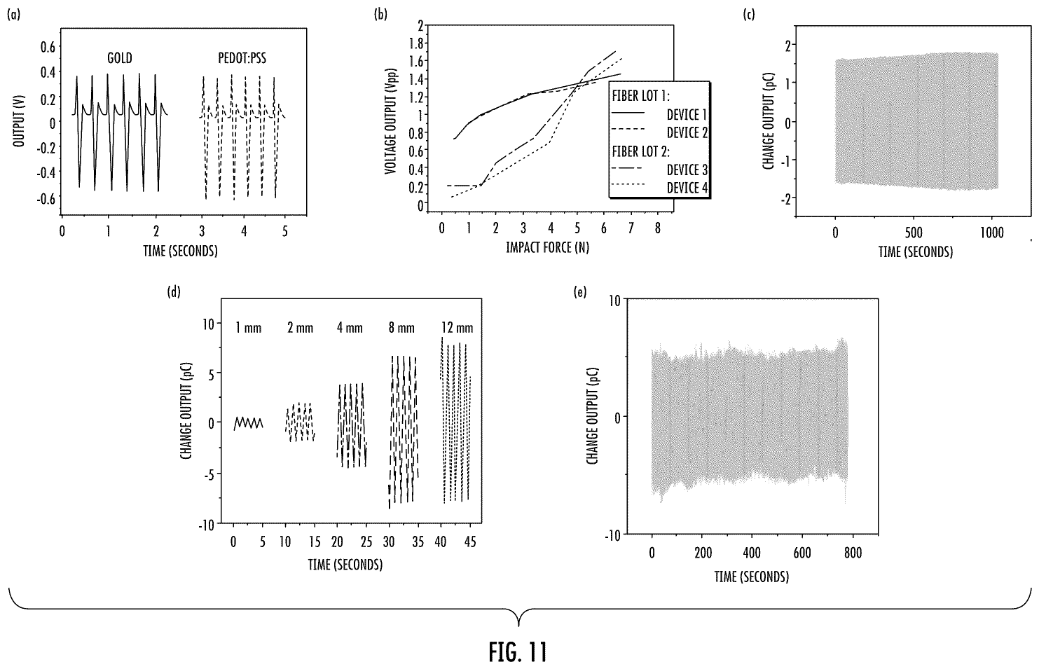

To determine the viability of piezoelectric sensor devices created through this inkjet printing process a 1 cm² square shaped electrode (schematic seen in the inset of FIG. 11(a)) was printed. For comparison, a standard PVDF-TrFE nanofiber device of the same size with magnetron sputtered gold electrodes (Cooke) and no PDMS infill was fabricated. FIG. 11(a) shows that a 1.25 N impact force at 3 Hz on the inkjet printed PEDOT:PSS device has a similar response as the standard gold electrode device of approximately 0.8 V peak-peak, demonstrating that the printed polymer electrode piezoelectric devices could show performances similar to that of standard metallic electrode piezoelectric nanofiber devices.

To determine the printed PEDOT:PSS single element's voltage sensitivity to force inputs, peak-to-peak voltage outputs were measured over a variety of forces from 0-7 N from direct impacts with a 1 cm² square impact area at a frequency of 1 Hz. The testing included two devices from two different fabrication lots of the PVDF-TrFE nanofibers. Device 1 and 2 were from fiber lot 1 and device 3 and 4 were from fiber lot 2. The testing set up included a shaker (2025E from the Modal Shop) for controlling of impact force and frequency, a force transducer (PCB Piezotronics), a fixture frame for mounting the devices, and a low-noise voltage preamplifier (Stanford Research Systems) with a gain of 1 and a low-pass filter cutoff of 30 kHz. FIG. 11(b) shows the voltage response of all four devices. Two fabrication lots of fibers were tested using two 1 cm² devices from each lot. Linear fits of the slope for device 1 and 2 of fiber lot 1 were 109 and 96 mVpp/N, respectively, and for devices 3 and 4 of fiber lot 2 were 254 and 256 mVpp/N, respectively. This shows that within a fiber lot devices perform similarly but due to lot variations, calibrations may be needed in the development of future force sensors using these materials. The standard deviation of the measured voltage values is represented for each device by the shading surrounding each line, while the standard deviation of each applied force is represented by the horizontal error bars. Based on the plot, the devices from the same fiber lot performed similarly, but there is a difference between fiber lots. A linear fit of the response of device 1 and 2, from fiber lot 1, was 109 mVpp/N and 96 mVpp/N with an R² of 0.924 and 0.921, respectively. A linear fit of the response of device 3 and 4, from fiber lot 2, was 254 mVpp/N and 256 mVpp/N with an R² of 0.971 and 0.959, respectively. These results suggest that variations in lot-to-lot fabrication of the PVDF-TrFE nanofibers can have an impact on the output of the force sensors, which may need a calibration step in order to accurately detect force. The piezoelectric response of one of these sensors was tested over a longer term of 1,000 cycles at a 1 Hz frequency and showed reasonable consistency (FIG. 11(c)).

To test the effect of bending on the piezoelectric response of the devices, a device with a 2 cm² substrate and a 1 cm² active area electrode was clamped to a fixture and the shaker was used to control linear displacement, causing the device to bend. The device was connected to a charge amplifier (Measurement Specialties) with a 100 pF feedback capacitor and a gain of 1. The signal from the amplifier was digitized by an analog to digital convertor (National Instruments) and processed in LabView (National Instruments). In FIG. 11(d), the five different compressive linear displacements tested, 1, 2, 4, 8, 12 mm are shown with their representative charge outputs. The charge output increased with increasing linear displacements to a maximum of 8.9 pC. A linear fit of the response of the device was found to be 0.687 pC/mm of linear displacement with an R² of 0.958. The response to bending was tested over the long-term of 800 cycles at a rate of 2 Hz (FIG. 11(e)) and held steady around 8 pC charge output at a linear displacement of 8 mm.

The developed method enables fabrication of all-polymer piezoelectric devices that are responsive to both bending and impact forces. The mechanical flexibility of the devices can allow use in biomedical applications, such as measuring pulse rate from near surface arteries. As a proof-of-concept a 1 cm² device was tested on a healthy 27-year-old male.

The left carotid artery was the first location tested, where the device was mounted with Kapton tape to the skin superficially to the artery. The mounted sensor had no external pressure applied other than the mounting tape. Outputs from the device were passed through a charge amplifier with a 100 pF feedback capacitor, and a low pass filter of 10 kHz. The amplifier was always set to a 40 dB gain and the data was scaled back during post processing. The test subject was in a relaxed, seated position and was asked to avoid movement and to perform various types of breathing exercises.

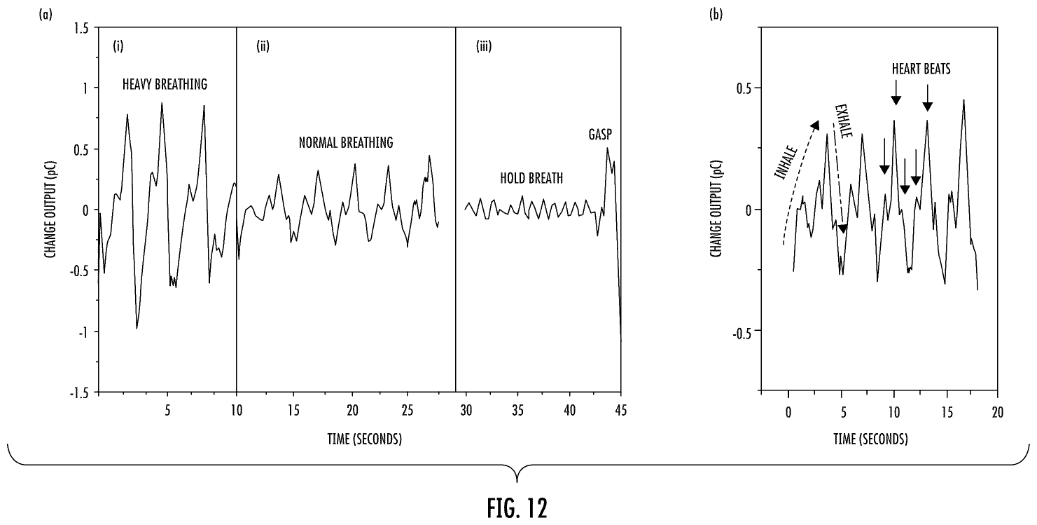

In FIG. 12(a), the effect of different types of breathing on the output of the device on the carotid artery are shown. FIG. 12(a) (i) shows the output from heavy breathing after exercise, while FIG. 12(a) (ii) shows the output from normal breathing. The shape and frequency of the waveform during normal breathing is similar to that of heavy breathing but of lower amplitude. The breathing rate is approximately 0.33 Hz during normal breathing and slightly faster during heavy breathing.

During both heavy and normal breathing, there are smaller peaks appearing at a frequency near 1 Hz that appear to correspond with the heart pulse. This is verified in FIG. 12(a) (iii) in which the breath is held, and the smaller amplitude peaks still appear at a nearly 1 Hz frequency (60 beats per minute), in the healthy range of the average adult. FIG. 12(a) (iii) also shows the change in output of the device during a gasp after the breath is held.

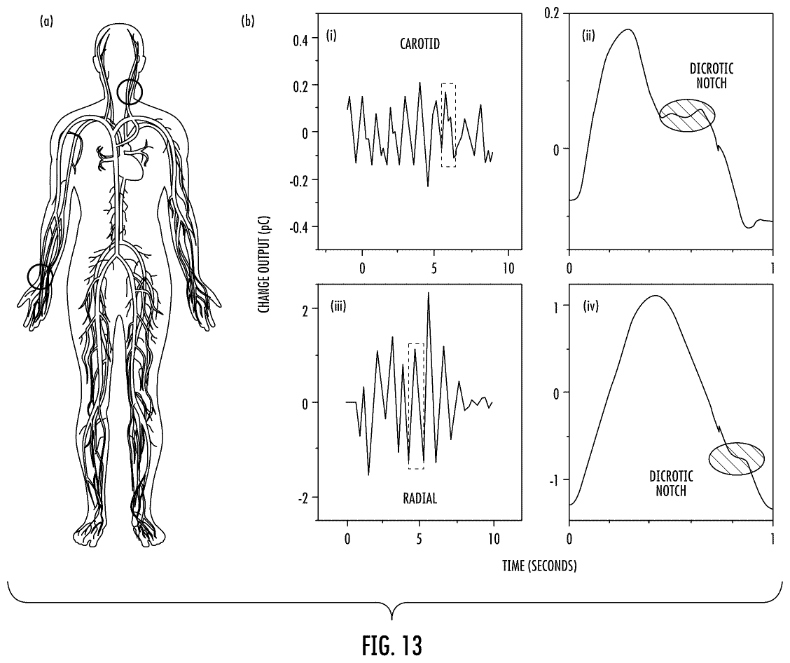

FIG. 12(b) shows a zoomed in version of the normal breathing in FIG. 12(a) (ii). At this breathing frequency, each breath has approximately three heartbeats and the difference between an inhale and exhale can be determined. The inhale is comprised of a lower slope increase, peaking at 0.3 pC, while the exhale is a much sharper decline, falling to −0.3 pC. The detection of the pulse waveform from the carotid artery can be used in applications because the waveform can offer clinically relevant measurements. The radial artery was also tested for the ability to detect the pulse waveform. FIG. 13(a) is a schematic showing the locations of the sensor placement, the left carotid artery and the right radial artery.

For the carotid artery, further analysis of the pulse output over a 10-second window in FIG. 13(b) (i) again verifies the pulse rate of approximately 60 beats per minute. Further analysis of a single pulse waveform over a 1-second time window is shown in FIG. 13(b) (ii). The full pulse waveform can be seen, with the maximum charge output due to the systolic pressure and the minimum charge output due to the diastolic pressure. The sensor is sensitive enough for the detection of the separation of systolic and diastolic phase, marked by the dicrotic notch.

The device was also tested on the radial artery, which has a noticeably weaker pulse. The use of a wrist band was needed to maintain a steady pressure on the device against the radial artery to increase signal output. No pulse was easily discernable without the applied pressure. However, after the wrist band was applied, an output of similar frequency (60 beats per minute) to the carotid artery placed device was found (FIG. 13(b) (iii)). The output from the radial artery was noticeably noisier than the carotid artery. In FIG. 13(b) (iii), the output amplitude is higher than that of the device on the carotid artery in FIG. 13(b) (i), which is due to the applied external pressure of the wrist pressure output by the radial artery. Similar to FIG. 13(b) (ii), in FIG. 13(b) (iv) a plot of a single pulse wave from the radial artery is shown. Here, the dicrotic notch is less distinguishable than in the carotid artery pulse.

The inkjet printed sensor can detect pulse rate from both the carotid and radial artery. The conformability of the device allows it to form to the skin more easily than standard a ceramic piezoelectric device.

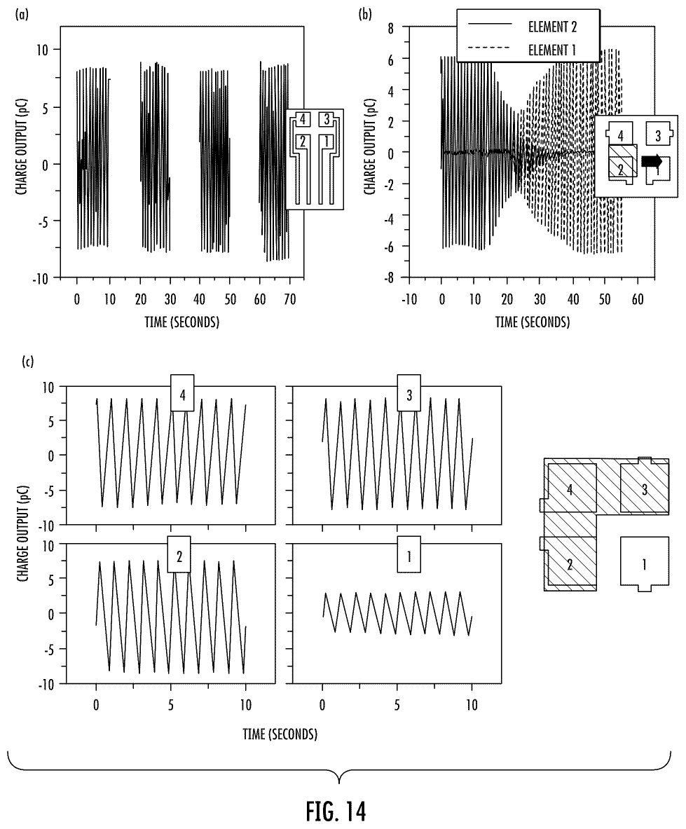

A fabrication method that utilizes an inkjet printer can provide rapid prototyping ability and easy pattern formation. To show application of this patterning ability, a 2×2 array was printed (schematic seen in the inset of FIG. 14(a)). The 2×2 array was made up of four 1 cm² square elements spaced 0.3 cm apart, all on the same PVDF-TrFE/PDMS composite substrate. FIG. 14(a) shows individual outputs of each element from a 5 N impact at 1 Hz. The test shows that each of the elements performs similarly to equal force inputs with outputs near 8 pC.

The array was also tested to determine the relationship between outputs of adjacent elements within the array. A 1 cm² acrylic square was impacted onto one of the elements, element 2, at a frequency of 1 Hz. The square was then slowly moved towards another element, element 1, and the output of both elements was measured. As shown in FIG. 14(b), while the elements were both under full impact of the square, they had similar outputs of 6 pC. While the square was impacted on the opposite element the output fell to 1/10 of the direct impact value, below 0.6 pC. While element 2 was directly under the impact it had an output of 6.15 pC versus 0.11 pC of element 1. While element 1 was directly under the impact it had an output of 6.55 pC versus 0.10 pC from element 2. This represented an average of a 60.7× increase in signal. The result suggests that the array structure can be used in applications with spatial sensitivity to determine location of various forces.

This was validated using various shapes applied to the array while testing the output of all four elements. Shapes tested were single 1 cm² squares, a 2×1 element-width rectangle, a square that covered all 4 elements at once, and an L-shape that covered three of the four elements. The L-shape was determined to be the most complex shape and in the inset of FIG. 14(c) is shown impacting elements 2, 3, and 4. FIG. 14(c) shows the outputs from each of the individual elements from a 1 Hz impact of the L-shape. Element 1 showed a much lower output compared to the other 3 elements, suggesting that these devices could be used for shape detection. The three elements being impacted by the device show similar outputs around 8 pC, while element 1, not impacted by the L-shape, shows an output of only 3 pC. This validates that the inkjet printed array structures can be used for shape detection and opens the sensor to further applications involving spatial sensitivity to force.

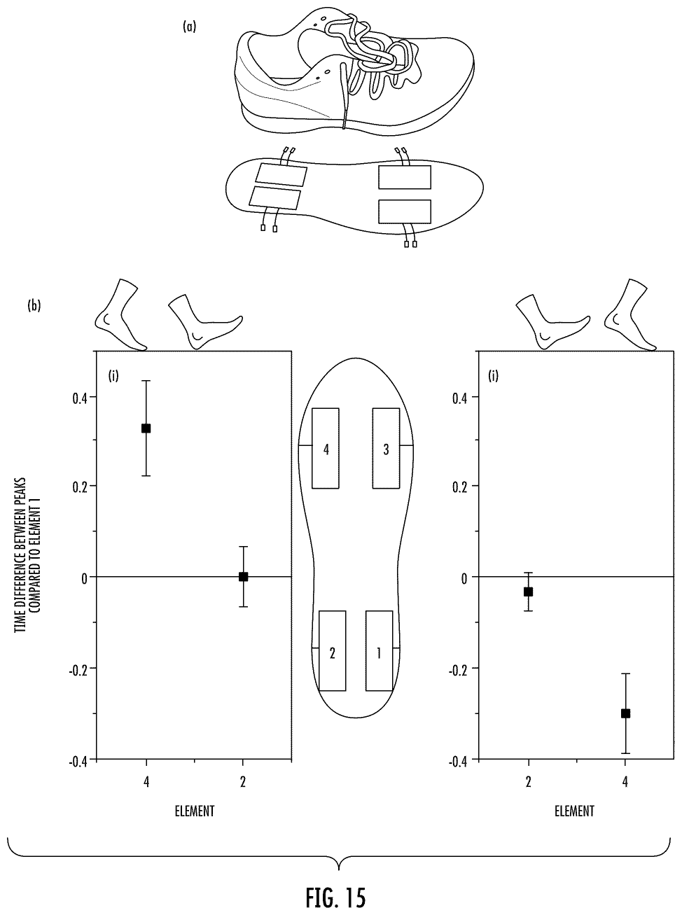

To further explore applications of the inkjet printed array structures, a 2×2 array of sensor to fit on the sole of a shoe was printed. A photograph of the shoe and the inkjet printed sensor array on the shoe sole can be seen in FIG. 15(a). Each element of the sensor array had an active area of 2 cm by 5 cm.

To test the ability of the sensor to determine spatiotemporal inputs, outputs from the four elements during a toe-to-heel step (FIG. 15(b) (i)) and a heel-to-toe step (FIG. 15(b) (ii)) were measured. The time value of the peak outputs from each element were subtracted from the time values of the peak outputs from element 1 at the right heel. In doing this the time difference between pressure inputs at various parts within the shoe can be determined.

In FIG. 15(b), the results of the time differences of outputs from element 4 (left front) and element 2 (left heel and next to element 1) are shown. During a toe-to-heel foot movement in (i), element 2 has a mean time difference of 0.003 seconds. This shows good agreement between both sensor elements in the heel. At the front of the foot, element 4, has a mean time difference of +0.331 seconds, which is expected because the front of the foot is striking the ground before the heel during a toe-to-heel movement and so the time difference between peak outputs from element 1 and 4 should be a positive number.

During a heel-to-toe foot movement in FIG. 15(b) (ii), element 2 has a mean time difference of −0.030 seconds, again in good agreement with element 1. At the front of the foot, element 4, has a mean time difference of −0.298 seconds. This again is expected, as the time value should be opposite of the toe-to-heel movement. This shows that the spatiotemporal difference between the front and back of the shoe can be determined, allowing determination the difference between a heel-to-toe or a toe-to-heel foot strike.

The devices disclosed herein are capable of measuring both the pulse rate and breath rate, and the heaviness of breathing appears to have an impact and may be measurable. The devices were also used for detecting the type of foot movement. These devices were fabricated using electrospinning and inkjet printing, which are both highly scalable fabrication methods. The ability to make use of inkjet printing of conductive polymer PEDOT:PSS electrodes on electrospun piezoelectric PVDF-TrFE nanofibers may allow for fabrication of easily-patternable all-polymer piezoelectric devices at large scale.

A benefit of a fabrication method for inkjet printing polymer electrodes on PVDF-TrFE nanofibers is the ability to rapidly fabricate a variety of electrode patterns. This allows development of devices for a variety of applications. With the ability of a single device to detect pulse waveforms in the wrist and neck, another array structure could offer the ability for further pulse wave analysis such as pulse wave velocity.

Through the addition of a physical separation layer of PDMS, inkjet patterning of PEDOT:PSS electrodes on PVDF-TrFE nanofibers can be performed to create an all-polymer piezoelectric array device. The fabrication methods were characterized, and as a proof of concept, single element and simple arrays were fabricated and characterized. The device's applications were demonstrated for use in cardiovascular sensing in the detection of both radial and carotid arterial pulses, as well as variations in the breath. Further array designs were also capable of detecting foot movement with spatiotemporal inputs. The fabrication method can be used as a highly scalable method for fabricating sensitive and flexible piezoelectric sensors.

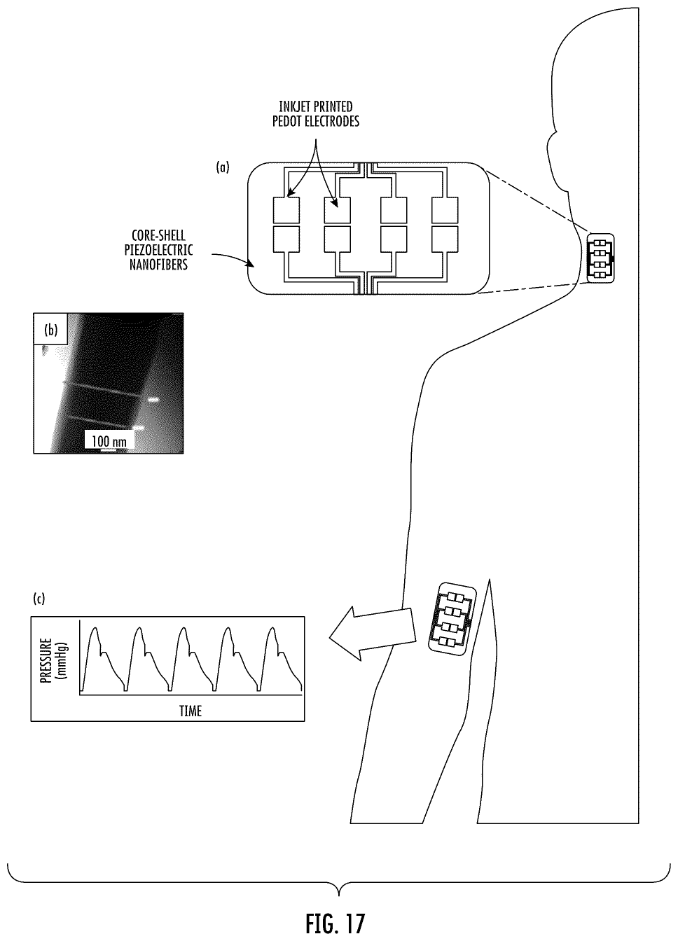

### Example 3 (FIGS. 16-17)

As shown in FIGS. 16 and 17, the sensitivity of the piezoelectric polymer pressure sensors can be improved to develop a highly-sensitive, piezoelectric patch for the detection of pulsatile pressure flows through the arteries. Two rigid ceramic PZT sensors have the signal to noise ratio (SNR), greater than 35 dB, capable of accurately determining PWV on a human wrist via the radial artery. An array structure of a piezoelectric material can be used for superficial arterial flow monitoring. When calculating PWV, the well-defined structure of the array can compensate for error in the PTT by eliminating error in the distance between sensors measurements, as well as offering a larger number of signals to derive data from.

To improve upon the sensitivity of flexible piezoelectric polymers, nanostructured fibers can be used. Core-shell piezoelectric nanofibers have a 4.5× greater sensitivity to pressure than standard aligned PVDF nanofibers, which were shown to have an 8.8× greater sensitivity than spin-cast PVDF thin films (FIG. 16). This makes them well-suited as highly sensitive and flexible material. To form array devices with these nanofibers, a method for iteratively patterning flexible electrodes is performed. For example, inkjet printing is performed.

A coaxial needle electrospinning method is used to fabricate the core-shell nanofiber structure. The electrospinning process for preparing PEDOT-PVDF core-shell nanofibers uses a 14 wt % PVDF-TrFE powder (70:30) dissolved in DMF-MEK (25:75). The PEDOT core solution is 4.307 g of poly(3,4-etylenedioxythiophene) poly(styrenesulfonate) (PEDOT:PSS, 2% in water, Sigma Aldrich) with 0.113 g polyethylene oxide (PEO, 600 kD) in 0.58 g DMF. The core-shell fiber is electrospun at 12 kV using a flow rate of 0.5 and 1.5 mL/h for the core and shell solutions, respectively. The final nanofiber is collected on an aluminum rotating drum collector. The electrospinning process typically yields fibers 0.1-1 μm in diameter. To connect the inner core PEDOT electrode, a solution etches through the outer PVDF shell layer, which is typically done using a silver paint, as its solvent acts as a good etchant for PVDF, but not for PEDOT:PSS. Lastly, an outer electrode is applied to the PVDF shell, which uses a selective deposition of copper or gold thin film from a magnetron sputter system or with inkjet printing in order to further improve the pattern-ability of the devices. These core-shell nanofiber structures showed a 4.5× greater sensitivity to pressure than standard PVDF nanofibers (FIG. 16).

The core-shell nanofibers are patterned with polymer electrodes to form a flexible piezoelectric array. The core-shell nanofiber structures has electrode array patterns deposited to form functional piezoelectric devices using inkjet printing (Fujifilm Dimatix DMP-2850). An inkjet printing method for the deposition and patterning of PEDOT:PSS conductive polymer electrodes on PVDF nanofiber matrixes can be used on the core-shell nanofibers, which is described in other examples. The first step is printing an electrode layer to etch through the PVDF shell layer to electrically connect to the inner PEDOT electrode. This conductive etching ink can be silver nanoparticle and PEDOT:PSS inks. Next, the porous fiber matrix is infilled with a thin layer of 1:1 diluted tert-Butyl alcohol (TBA):PDMS. The PEDOT:PSS ink is then inkjet printed on the surface in the desired electrode pattern, easily altered for any desired shape or pattern.

Each individual element is tested for its force and frequency response using a shaker. To validate the ability to detect a pressure pulse wave, spatiotemporal sensitivity to force is analyzed on the entire array structure through the use of a shaker applying a known force to a specified area of the array. Through this method, force location sensitivity of each element in the array and interference due to crosstalk between elements can be determined. The Young's modulus and other important mechanical properties of the fully fabricated array device also can be analyzed using a tensile test (Instron). Using cyclic bending, the effects of long term bending on the functionality of the device can be tested, validating its use as a flexible sensor.

### Example 4 (FIG. 18)

For those with hypertension (HTN), there is a need for the continuous monitoring of their BP. To meet this need, a highly flexible patch for wearable continuous BP and cardiovascular monitoring that is both low-power and conformable to the skin can be used. Using highly scalable fabrication methods including inkjet printing and electrospinning, an array of pressure sensors using flexible piezoelectric materials that require little to no power to function can be fabricated. Flexible printed circuit boards (PCB) can be achieved by integrating and printing small electrical components on a flexible thin film. The ability to selectively pattern these materials enables sensor arrays to detect vital physiological signals in the form of heart rate (HR), arterial pulse wave velocity, and BP, which was tested on the carotid and radial arteries from the skin surface. The technology allows fabrication of pressure sensors in any shape and have them be conformable to the human skin. This design can allow for wearable continuous monitoring of HR and BP, the latter that is not currently available on the market. This can provide users, such as those with HTN, a means for continuously monitoring their cardiovascular health outside of the doctor's office. Due to white coat hypertension and infrequent doctor visits, BPs obtained in a doctor's office are not an ideal means to monitor HTN. Ambulatory BPs have been a proposed solution but they only track BPs for 24 hours. Having continuous BP monitoring for years would be an important advance in this field.

The system in FIG. 18 is a wearable sensor using piezoelectric polymers with engineered nano- and micro-structures. The device is ultra-flexible, and its mechanical properties can match those of human skin to provide extraordinary user experience. The device is capable of continuously monitoring vital physiological signals such as HR and BP continuously throughout the day with minimum power consumption owing to the intrinsic properties of the material and fabrication technology. The sensor can use one of the embodiments disclosed herein.

For those suffering from HTN, there is a lack of available devices capable of continuously monitoring HR and BP that is wearable, flexible, and long lasting. An ultra-flexible wearable device with great comfortability that can continuously monitor and record HR and BP with minimum power requirements is an improvement for users. This device can monitor signals continuously owing to its low power consumption compared to other wearable devices that can only take a limited number of measurements everyday due to their high-power consumption and limited battery life. As a result, this device can continuously monitor the wearer's vital signals for every second throughout the day without the need to charge the battery, providing a comprehensive dataset for the user or physician to review and analyze. Second, the device is ultra-flexible and conformable to the skin. Rigid electronics, which limit the ultimate form factor of the device, can be minimized or avoided. This device, built with soft materials, shares a similar mechanical property with that of skin, providing a comfortable wearing experience.

This device can be used by those who have underlying cardiovascular diseases, such as HTN and atrial fibrillation. These people need to check vital signs frequently for early alert. In addition, continuous monitoring throughout the day, month, and year can provide abundant data to their healthcare providers for better treatment. This device also can be used by fitness-conscious users. This device is low profile, extremely flexible, and conformal to the skin, so it can be used in a discrete manner for those who wish to gain a better understanding of how their BP changes during exercise.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

What is claimed is:

1. A system comprising:
piezoelectric nanofibers;
an encapsulation polymer, wherein the encapsulation polymer is configured to fill voids between the piezoelectric nanofibers; and
patterned electrodes in an array, wherein the system has a thickness from 5 μm to 1 mm.

2. The system of claim 1, further comprising a processor in electronic communication with the patterned electrodes.

3. The system of claim 1, further comprising a wireless data transmission system in electronic communication with the patterned electrodes.

4. The system of claim 1, wherein the piezoelectric nanofibers are poly(vinylidene fluoride-cotrifluoroethylene).

5. The system of claim 1, wherein the piezoelectric nanofibers and/or the encapsulation polymer include a dopant.

6. The system of claim 5, wherein the dopant is carbon nanotubes, lead zirconate titanate (PZT), barium titanate, and/or zinc oxide.

7. The system of claim 1, wherein the encapsulation polymer is polydimethylsiloxane or parylene.

8. The system of claim 1, wherein from 90% to 100% of the voids are filled with the encapsulation polymer.

9. The system of claim 1, wherein the patterned electrodes are poly(3,4-ethylenedioxythiophene) polystyrene sulfonate.

10. The system of claim 1, wherein the system has a Young's modulus is from 360 kPa to 870 kPa.

11. The system of claim 1, wherein the piezoelectric nanofibers, the encapsulation polymer, and the patterned electrode are disposed on a substrate.

12. The system of claim 1, wherein the piezoelectric nanofibers, the encapsulation polymer, and the patterned electrode are configured in a core with a shell.

13. The system of claim 12, wherein the patterned electrodes are in the core and the piezoelectric nanofibers are disposed around the core in the shell.

14. The system of claim 1, wherein the encapsulation polymer has a thickness from 10 μm to 25 μm.

15. The system of claim 1, wherein the piezoelectric nanofibers have a thickness of approximately 50 μm and the encapsulation polymer has a thickness on either side of the piezoelectric nanofibers of 15 μm.

16. The system of claim 1, wherein the piezoelectric nanofibers have a diameter from 10 nm to 10 μm.

17. A method of measuring blood pressure using the system of claim 1, wherein an applied pressure produces a strain in the piezoelectric nanofibers thereby producing a charge that is processed.

18. A method of measuring pressure applied to the system using the system of claim 1, wherein an applied pressure produces a strain in the piezoelectric nanofibers thereby producing a charge that is processed.
